(19) United States
(12) Patent Application Publication
(10) Pub. No.: US 2023/0076608 A1
(43) Pub. Date: Mar. 9, 2023

(54) FASTER FITTED Q-ITERATION USING ZERO-SUPPRESSED DECISION DIAGRAM

(71) Applicant: INTERNATIONAL BUSINESS MACHINES CORPORATION, Armonk, NY (US)

(72) Inventor: Hiroshi Kajino, Tokyo (JP)

(21) Appl. No.: 17/467,615

(22) Filed: Sep. 7, 2021

(51) Int. Cl. G06F 30/27; G06N 7/00

(52) U.S. Cl. CPC G06F 30/27; G06N 7/00

(57) ABSTRACT

A computer-implemented method for estimating a state-action value function for a Fitted Q-iteration is provided including obtaining a set of tuples D and a discount factor γ, each of the set of tuples including a state s, an action a, a reward r, and a resulting state s′, constructing a zero-suppressed decision diagram (ZDD) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′, updating parameters w∈ [symbol] ^{D}, θ of a state-action value function Q (s, a; w, θ); and repeating the updating step a predetermined times by incrementing t.

### BACKGROUND

The present invention relates generally to machine learning, and more specifically, to methods and systems for faster Fitted Q-iteration using a zero-suppressed decision diagram.

Q-learning is a popular reinforcement learning algorithm, with several applications in automation and robotics. Reinforcement learning algorithms compute control policies by means of an agent, who can learn directly on the system (on-line control) or from an interaction with a simulator of the system (off-line or batch control). The desired control policy is such that, from any initial state, it chooses actions that maximize the reward accumulated over time by the agent. Q-learning uses an action-value model, which creates a function to deal with different states. Q-learning was conceived to determine the optimal policy in a step-by-step manner.

Though Q-learning with full state representation has been shown to be convergent, when the state of the environment is partially observable (for example imprecisions or delays of sensor devices), then suitable approximation methods are required for discovering the optimal policy. For finite and small enough state and action spaces, the Q-function can be represented in tabular form, so its approximation (in batch and in on-line mode) and the derived control policy are straightforward. This approach, however, cannot be used successfully when dealing with continuous or very large discrete state and/or action spaces.

### SUMMARY

In accordance with an embodiment, a computer-implemented method for estimating a state-action value function for a fitted Q iteration is provided. The computer-implemented method includes obtaining a set of tuples D and a discount factor γ, each of the set of tuples including a state s, an action a, a reward r, and a resulting state s′, constructing a zero-suppressed decision diagram (ZDD) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′, updating parameters w∈ [symbol] ^{D}, θ of a state-action value function Q(s, a; w, θ), and repeating the updating step a predetermined times by incrementing t.

In accordance with another embodiment, a computer program product for estimating a state-action value function for a fitted Q iteration is provided. The computer program product includes a computer readable storage medium having program instructions embodied therewith, the program instructions executable by a computer to cause the computer to obtain a set of tuples D and a discount factor γ, each of the set of tuples including a state s, an action a, a reward r, and a resulting state s′, construct a zero-suppressed decision diagram (ZDD) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′, update parameters w∈ [symbol] ^{D}, θ of a state-action value function Q(s, a; w, θ), and repeat the updating step a predetermined times by incrementing t.

In accordance with yet another embodiment, a system for estimating a state-action value function for a fitted Q iteration is provided. The system includes a memory and one or more processors in communication with the memory configured to obtain a set of tuples D and a discount factor γ, each of the set of tuples including a state s, an action a, a reward r, and a resulting state s′, construct a zero-suppressed decision diagram (ZDD) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′, update parameters w∈ [symbol] ^{D}, θ of a state-action value function Q(s, a; w, θ), and repeat the updating step a predetermined times by incrementing t.

In accordance with another embodiment, a computer-implemented method for estimating a state-action value function for a fitted Q iteration is provided. The computer-implemented method includes obtaining a set of tuples D and a discount factor γ, each of the set of tuples including a state s, an action a, a reward r, and a resulting state s′, constructing a binary decision diagram (BDD) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′, updating parameters w∈ [symbol] ^{D}, θ of a state-action value function Q(s, a; w, θ), and repeating the updating step a predetermined times by incrementing t.

In accordance with yet another embodiment, a computer-implemented method for estimating a state-action value function for a fitted Q iteration is provided. The computer-implemented method includes obtaining a set of tuples D and a discount factor γ, each of the set of tuples including a state s, an action a, a reward r, and a resulting state s′, constructing a plurality of zero-suppressed decision diagram (ZDDs) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′, updating parameters w∈ [symbol] ^{D}, θ of a state-action value function Q(s, a; w, θ), and repeating the updating step a predetermined times by incrementing t.

It should be noted that the exemplary embodiments are described with reference to different subject-matters. In particular, some embodiments are described with reference to method type claims whereas other embodiments have been described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be described within this document.

These and other features and advantages will become apparent from the following detailed description of illustrative embodiments thereof, which is to be read in connection with the accompanying drawings.

### BRIEF DESCRIPTION OF THE DRAWINGS

The invention will provide details in the following description of preferred embodiments with reference to the following figures wherein:

FIG. 1 is a block/flow diagram of an exemplary breakdown of machine learning in artificial intelligence (AI) and where Fitted Q-Iteration fits in, in accordance with an embodiment of the present invention;

FIG. 2 illustrates a method for implementing the Fitted Q-Iteration with the zero-suppressed decision diagram (ZDD), in accordance with an embodiment of the present invention;

FIG. 3 illustrates a practical application for the machine learning workflow for computational material discovery, in accordance with an embodiment of the present invention;

FIG. 4 illustrates a method for employing the Fitted Q-Iteration with the ZDD, in accordance with an embodiment of the present invention;

FIG. 5 is an algorithm for employing the Fitted Q-Iteration with the ZDD, in accordance with an embodiment of the present invention;

FIG. 6 is a block/flow diagram of an exemplary practical application for chemical discovery on how chemical properties are predicted or how new molecules are generated, in accordance with an embodiment of the present invention;

FIG. 7 is a block/flow diagram of an exemplary processing system for employing the Fitted Q-Iteration with the ZDD, in accordance with an embodiment of the present invention;

FIG. 8 illustrates practical applications for employing the Fitted Q-Iteration with the ZDD via an artificial intelligence (AI) accelerator chip, in accordance with an embodiment of the present invention;

FIG. 9 is a block/flow diagram of an exemplary cloud computing environment, in accordance with an embodiment of the present invention; and

FIG. 10 is a schematic diagram of exemplary abstraction model layers, in accordance with an embodiment of the present invention.

Throughout the drawings, same or similar reference numerals represent the same or similar elements.

### DETAILED DESCRIPTION

Embodiments in accordance with the present invention provide methods and devices for faster Fitted Q-iteration using a zero-suppressed decision diagram in offline reinforcement learning. Reinforcement learning aims to determine an optimal control policy from interaction with a system or from observations gathered from a system. In batch mode, it can be achieved by approximating the so-called Q-function based on a set of four-tuples (xₜ,uₜ, rₜ, xₜ₊₁) where xₜ denotes the system state at time t, uₜ the control action taken, rₜ the instantaneous reward obtained and xₜ₊₁ the successor state of the system, and by determining the control policy from this Q-function. The Q-function approximation may be obtained from the limit of a sequence of (batch mode) supervised learning problems. For finite and small enough state and action spaces, the Q-function can be represented in tabular form, so its approximation (in batch and in on-line mode) and the derived control policy are straightforward. This approach, however, cannot be successfully used when dealing with continuous or very large discrete state and/or action spaces.

In order to respond to these issues, the Fitted Q-iteration (FQI) algorithm was introduced. FQI is a batch mode reinforcement learning algorithm which yields an approximation of the Q-function corresponding to an infinite horizon optimal control problem with discounted rewards, by iteratively extending the optimization horizon. At the first iteration, the FQI algorithm uses the training set, with inputs as the state-action pairs and outputs as the observed rewards, to produce an approximation of the expected reward. In the subsequent iterations, only the output values are updated using the value of the Q-function produced at the preceding step and information about the reward and the successor state reached in each tuple. Since all updates are done offline, approximating the Q-function can be viewed as a separate, supervised learning problem. The question arises if there are function approximators especially suited for offline updating.

The idea of FQI was derived from the pioneer work of Ormoneit and Sen, who combined the idea of fitted value iteration with kernel based reinforcement learning, and reformulates the Q-function determination problem as a sequence of kernel-based regression problems. FQI was introduced by Ernst to fit (using a set of four-tuples) any (parametric or non-parametric) approximation architecture to the Q-function.

However, FQI alone may not be sufficient for certain applications, such as, e.g., the discovery of high-performance functional materials or computational material discovery.

Finding new materials with good performance is the eternal theme in materials science. Currently, experimental and computational screenings for new materials discovery involve element replacement and structure transformation. However, the compositional search space, structural search space, or both tend to be sharply constrained. Both screening methods may also require massive amounts of computation or experimentation and usually result in effort being directed in incorrect directions in an “exhaustive search,” which consumes considerable time and resources. In consideration of this fact and the advantages of machine learning, a method combining machine learning with computational simulation is proposed for the evaluation and screening of new materials to provide suggestions for new and better materials.

The exemplary embodiments of the present invention alleviate such issues in finding new materials by implementing a faster Fitted Q-iteration by using a zero-suppressed decision diagram (ZDD). Such configuration can be beneficial in, e.g., computational material discovery for generating new molecular structures satisfying target property values.

It is to be understood that the present invention will be described in terms of a given illustrative architecture; however, other architectures, structures, substrate materials and process features and steps/blocks can be varied within the scope of the present invention. It should be noted that certain features cannot be shown in all figures for the sake of clarity. This is not intended to be interpreted as a limitation of any particular embodiment, or illustration, or scope of the claims.

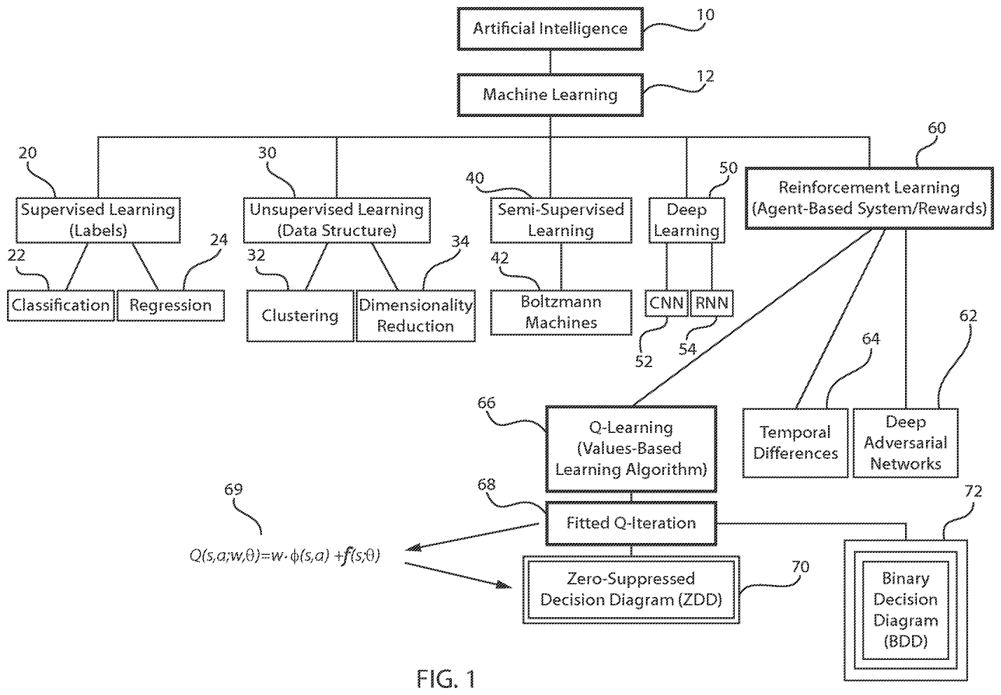

FIG. 1 is a block/flow diagram of an exemplary breakdown of machine learning in artificial intelligence (AI) and where Fitted Q-Iteration fits in, in accordance with an embodiment of the present invention.

Artificial intelligence 10 includes machine learning 12. Machine learning 12 can be divided into supervised learning 20, unsupervised learning 30, semi-supervised learning 40, deep learning 50, and reinforcement learning 60.

Supervised learning 20 can include, e.g., classification 22 and regression 24.

Unsupervised learning 30 can include, e.g., clustering 32 and dimensionality reduction 34.

Semi-supervised learning 40 can include, e.g., application of Boltzmann machines 42.

Deep learning 50 can include, e.g., convolutional neural networks (CNN) 52 and recurrent neural networks (RNN) 54.

Reinforcement learning 60 can include temporal differences 64 and deep adversarial networks 62. Reinforcement learning 60 can further include Q-learning 66, which includes the Fitted Q-Iteration 68 combined with the zero-suppressed decision diagram 70 of the exemplary embodiments of the present invention. The Fitted Q-Iteration 68 combined with the zero-suppressed decision diagram 70 can implement Q(s, a; w, θ)=w·ϕ(s, a)+f(s; θ) designated as equation 69, where ϕ(s, a) ∈ {0,1}^{D} is a sparse bit vector and w ∈ [symbol] ^{D} and θ are learnable parameters.

In an alternative embodiment, the Fitted Q-Iteration 68 can be combined with the Binary Decision Diagrams (BDD) 72. Binary decision diagrams (BDDs) provide a compact way to uniquely represent a given Boolean function. A BDD is a rooted, directed, acyclic graph consisting of decision nodes and terminal nodes.

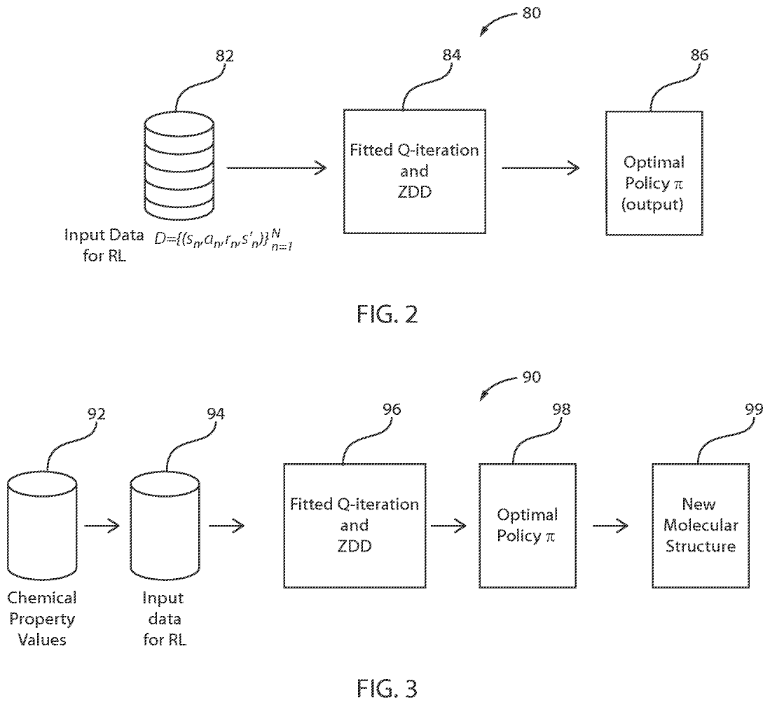

FIG. 2 illustrates a method for implementing the Fitted Q-Iteration with the zero-suppressed decision diagram (ZDD), in accordance with an embodiment of the present invention.

In the offline reinforcement learning diagram 80, the input data 82 is fed into the Fitted Q-Iteration with the zero-suppressed decision diagram (ZDD) component 84 to obtain an optimal policy 86. The input data can be designated as [symbol] ={(sₙ, aₙ,rₙ, s′ₙ)}ₙ₌₁^{N}, where s ∈ [symbol] : state, a ∈ [symbol] (s): action of agent on state s, r=r(s, a) ∈ [symbol] : reward of action a on state s, and s′=T (s, a): the next state.

Traditionally, reinforcement learning (RL) is thought of as a paradigm for online learning, where the interaction between the RL agent and its environment is of fundamental concern for how the agent learns. In offline RL (known as batch RL), the agent learns from a fixed-sized dataset, collected by some arbitrary and possibly unknown process Eliminating the need to interact with the environment is noteworthy as data collection can often be expensive, risky, or otherwise challenging, particularly in real-world applications. Consequently, offline RL enables the use of previously logged data or leveraging an expert, such as a human operator, without any of the risk associated with an untrained RL agent. However, the main benefit of offline RL, the lack of environment interaction, is also what makes it a challenging task.

In the present case, the difficulty with Fitted Q-Iteration is that it requires max operation over [symbol] (s) which is time consuming when | [symbol] (s)| is large:

$${θ}_{t + 1} = {argmin}_{θ} {\sum }_{( s , a , {s}^{′} , r ) ∈ 𝒟} {[ \begin{matrix}Q ( s , a ; θ ) \\ - ( r + γ \cdot {max}_{{a}^{′} ∈ 𝒜 ( {s}^{′} )} Q ( {s}^{′} , {a}^{′} ; {θ}_{t} ) )\end{matrix} ]}^{2}$$

where γ∈(0,1] and is a discounting factor.

The exemplary embodiments resolve such issue by incorporating a zero-suppressed decision diagram (ZDD) with the Fitted Q-Iteration.

In particular, an action value function is parameterized by:

Q(s,a;w,θ)=w·ϕ(s,a)+f(s;θ)

where ϕ(s, a) ∈ {0,1}^{D} is a sparse bit vector and w ∈ [symbol] ^{D} and θ are learnable parameters.

A zero-suppressed decision diagram (ZDD) is a compact data structure of sparse bit vectors. Given a ZDD of a set of bit vectors V ⊂{0,1}^{D} and weight w ∈ [symbol] ^{D},

$${max}_{v ∈ 𝒱} w \cdot v$$

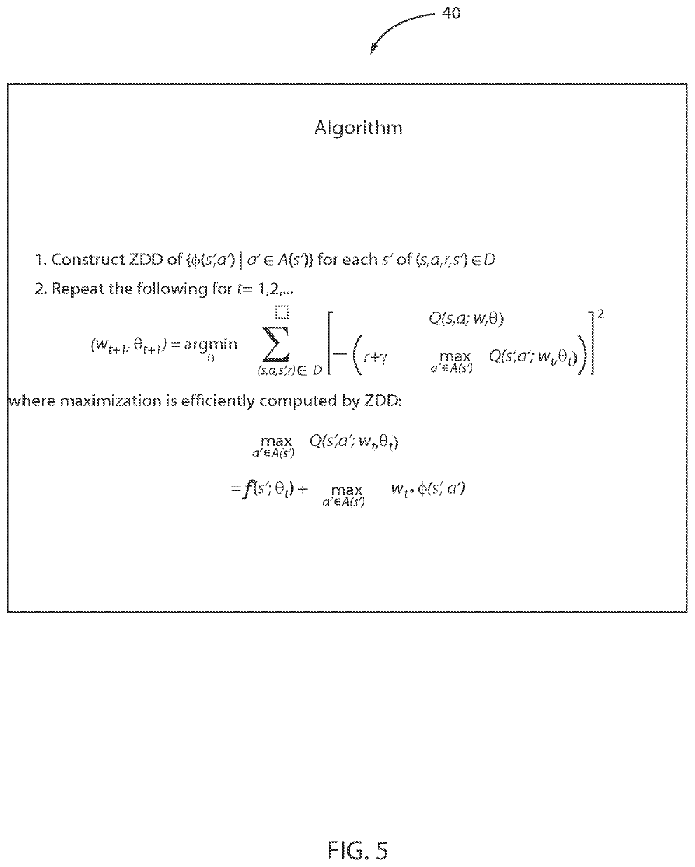

can be computed in computation time proportional to the size of ZDD, which is often much smaller than |V|. The algorithm is provided in FIG. 5 below.

Regarding ZDDs, ZDD is a particular kind of binary decision diagram (BDD) with fixed variable ordering. This data structure provides a canonically compact representation of sets, particularly suitable for certain combinatorial problems. A node in a ZDD is removed if its positive edge points to the terminal node 0. This provides an alternative strong normal form with improved compression of sparse sets.

When BDDs are applied to combinatorial problems, it may be observed that most of the positive edges of the decision nodes simply point to the 0-terminal. This may especially be true for matching strings. In these cases, a zero-suppressed binary decision diagram (ZSDD, ZBDD, or ZDD) may perform better than a standard BDD. A ZDD is a type of BDD designed to encode sets of combinations or a family of sets of primitive elements. A ZDD is a rooted, directed, acyclic graph (DAG) that includes terminal and non-terminal nodes. Each of the non-terminal nodes is labeled with a variable and has two outgoing edges to child nodes referred to as a negative edge (or LO edge) and positive edge (or HI edge).

Similar to standard BDDs, ZDDs have two terminal or leaf nodes labeled FALSE and TRUE (or 0-terminal and 1-terminal) which do not have outgoing edges. Further, the universe of all variables (or primitive elements) is ordered, and the order of the variables appearing on the nodes of any path through the ZDD is consistent with the total order. Additionally, each path through the ZDD that ends at the TRUE terminal node defines a set of variables in the family of sets.

FIG. 3 illustrates a practical application for the machine learning workflow for computational material discovery, in accordance with an embodiment of the present invention.

Regarding computational material discovery, traditionally, experiments used to play the key role in finding and characterizing new materials. Experimental research must be conducted over a long time period for an extremely limited number of materials, as it imposes high requirements in terms of resources and equipment. Owing to these limitations, important discoveries happened mostly through human intuition. A first computational revolution in materials science was fueled by the advent of computational methods, especially density functional theory (DFT), Monte Carlo simulations, and molecular dynamics, that allowed researchers to explore the phase and composition space far more efficiently. In fact, the combination of both experiments and computer simulations has allowed to cut substantially the time and cost of materials design. The constant increase in computing power and the development of more efficient codes also allowed for computational high-throughput studies of large material groups in order to screen for the ideal experimental candidates. These large-scale simulations and calculations together with experimental high-throughput studies are producing an enormous amount of data making possible the use of machine learning methods to materials science.

The availability of large datasets combined with the improvement in algorithms and the exponential growth in computing power led to an unparalleled surge of interest in the topic of machine learning. As these algorithms start to find their place, they are heralding a second computational revolution. Because the number of possible materials is estimated to be as high as a googol (10¹⁰⁰), this revolution is doubtlessly required.

Referring back to FIG. 3, in the offline reinforcement learning diagram 90, molecule structures and chemical property values 92 are used as a dataset as input data 94 for the offline RL. The extracted chemical features are processed by a chemical optimization procedure which includes the Fitted Q-Iteration with the zero-suppressed decision diagram (ZDD) 96 to obtain an optimal policy 98 resulting in the generation of new molecular structures 99.

Therefore, machine learning provides a new means of screening novel materials with good performance, developing quantitative structure-activity relationships (QSARs) and other models, predicting the properties of materials, discovering new materials and performing other materials-related studies. One exemplary method of machine learning involves Fitted Q-Iteration with the zero-suppressed decision diagram (ZDD) 96, as described herein, for computation material discovery (CMD).

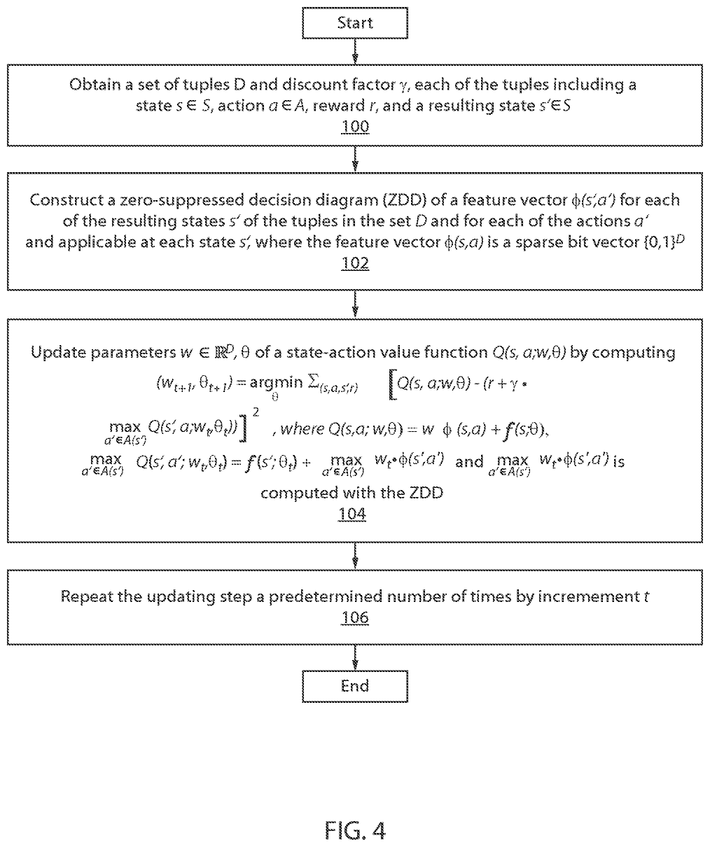

FIG. 4 illustrates a method for employing the Fitted Q-Iteration with the ZDD, in accordance with an embodiment of the present invention.

At block 100, obtain a set of tuples D and discount factor γ, each of the tuples including a state s ∈ S, action a ∈ A, reward r, and a resulting state s′ ∈ S.

At block 102, construct a zero-suppressed decision diagram (ZDD) of feature vectors {ϕ(s′, a)|a∈ [symbol] (s′)} for each of the resulting states s′ of the tuples in the set D, where the feature vector ϕ(s, a) is a sparse bit vector {0,1}^{D} and [symbol] (s′) is the set of actions applicable at state s′.

At block 104, update parameters w∈ [symbol] ^{D}, θ of a state-action value function Q(s, a; w, θ) by computing is

$$( {w}_{t + 1} , {θ}_{t + 1} ) = {argmin}_{θ} {\sum }_{( s , a , {s}^{′} , r )} [ Q ( s , a ; w , θ ) - ( r + γ \cdot {{max}_{{a}^{′} ∈ A ( {s}^{′} )} Q ( {s}^{′} , {a}^{′} ; {w}_{t} , {θ}_{t} ) ) ]}^{2} , \\ where Q ( s , a ; w , θ ) = w \cdot ϕ ( s , a ) + f ( s ; θ ) , \\ {max}_{{a}^{′} ∈ A ( {s}^{′} )} Q ( {s}^{′} , {a}^{′} ; {w}_{t} , {θ}_{t} ) = f ( {s}^{′} ; {θ}_{t} ) + {max}_{{a}^{′} ∈ A ( {s}^{′} )} {w}_{t} \cdot ϕ ( {s}^{′} , {a}^{′} ) \\ and {max}_{{a}^{′} ∈ A ( {s}^{′} )} {w}_{t} \cdot ϕ ( {s}^{′} , {a}^{′} )$$

computed with the ZDD.

At block 106, repeat the updating step a predetermined number of times by increment t.

Therefore, the assumption on the action-value function is:

Q(s,a;w,θ)=w·ϕ(s,a)+f(s;θ)

where ϕ is the feature vector of a state S and action a applicable at state s. The state is the molecule and the action is a modification applied to the molecule. The exemplary embodiments use the Morgan fingerprint (bit vector) for ϕ. Molecular fingerprints are a way of encoding the structure of a molecule. The most common type of fingerprint is a series of binary digits (bits) that represent the presence or absence of particular substructures in the molecule. Comparing fingerprints allows the determination of the similarity between two molecules to find matches to a query substructure, etc. The Morgan fingerprint is basically a reimplementation of the extended connectivity fingerprint (ECFP). Extended-Connectivity Fingerprints (ECFPs) are circular topological fingerprints designed for molecular characterization, similarity searching, and structure-activity modeling.

The main properties of ECFPs are that they represent molecular structures by means of circular atom neighborhoods, they can be very rapidly calculated, their features represent the presence of particular substructures, they are not predefined and can represent a huge number of different molecular features (including stereochemical information), they are designed to represent both the presence and the absence of functionality, since both are crucial for analyzing molecular activity, and their generation method can be flexibly customized to produce various types of circular fingerprints for diverse applications.

Returning back to the action-value function, now ZDD can be combined with such specific action-value function, the ZDD being a data structure for a set of sparse bit vectors. The combination of the specific action-value function and the ZDD enables compact representation supporting several operations and faster max/min for any element-wise weights.

As a result, at state sₜ, if the agent wishes to stop, the environment halts and returns the property of molecule sₜ. Otherwise, given the action aₜ, the environment applies the chemical reaction aₜ to the current molecule sₜ to derive the next state sₜ₊₁, which is the product of the chemical reaction. A chemical reaction can yield multiple candidates of products. In that case, the exemplary embodiments regard the candidate that has the lowest synthetic accessibility score as the product.

FIG. 5 is an algorithm 110 for employing the Fitted Q-Iteration with the ZDD, in accordance with an embodiment of the present invention.

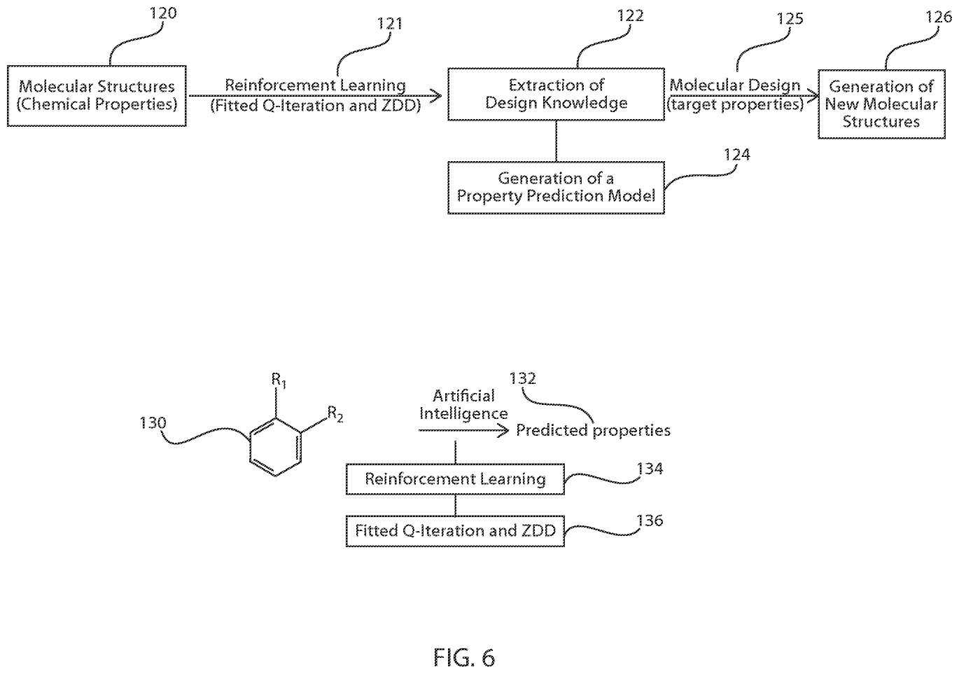

FIG. 6 is a block/flow diagram of an exemplary practical application for chemical discovery on how chemical properties are predicted or how new molecules are generated, in accordance with an embodiment of the present invention.

In one practical application, related to material discovery, molecular structures 120 having chemical properties can be used as a dataset to generate a property prediction model 124 via extraction of design knowledge 122. This can be accomplished by employing offline RL 121 with the Fitted Q-Iteration with the ZDD. Molecular designs 125 with target properties can enable the generation of new molecular structures 126. Thus, one example is shown, where artificial intelligence is applied to a molecular structure 130 to generate predicted properties 132. The artificial intelligence applied can be via offline RL 134 via the Fitted Q-Iteration with the ZDD 136.

In alternative embodiments, the agent recommends a set of items and the action is a feasible item combination.

In further alternative embodiments, a text-based action space is provided where the agent interacts with the environment by using texts. The action can be a grammatically correct sentence.

In further alternative embodiments, a graph-based action space is provided where the action is selecting a path in a graph to enable path planning and inference on a knowledge graph.

The benefits of all the embodiments include at least faster implementation of fitter Q Iteration, especially when | [symbol] (s′)| is very large.

To summarize, the properties of materials, such as hardness, melting point, ionic conductivity, glass transition temperature, molecular atomization energy, and lattice constant, can be described at either the macroscopic or microscopic level. There are two common methods of studying materials properties, that is, computational simulation and experimental measurement. These two methods involve complicated operations and experimental setup. Therefore, it is quite difficult to build computational simulations that fully capture the complicated logical relationships between the properties of a material and their related factors, and some of these relationships may even be unknown. Moreover, the experiments that are performed to measure the properties of compounds generally occur in the later stages of materials selection.

Consequently, if the results are not satisfactory, the enormous amounts of time and experimental resources invested up to that point prove to have been wasted. In addition, in many cases, it is difficult or nearly impossible to study the properties of materials even through massive computational or experimental efforts. Therefore, there is an urgent need to develop intelligent and high-performance prediction models that can correctly predict the properties of materials at a low temporal and computational cost. Machine learning concerns the construction and study of algorithms that can learn patterns from data. The basic idea of using machine learning methods for material property prediction is to analyze and map the relationships (nonlinear in most cases) between the properties of a material and their related factors by extracting knowledge from existing empirical data. FIGS. 1-6 show the fundamental framework for the application of machine learning in material property prediction by employing Fitted Q-Iteration with the ZDD.

In particular, large action space is a common issue in reinforcement learning, and a number of approaches have been developed so as to alleviate it. These approaches can be used for both online and offline settings, however, there are few methods focusing on the offline setting. The inventiveness of exemplary embodiments comes from the offline RL setting and the specific action value function of the form Q(s, a; w, θ)=w·ϕ(s, a)+f (s; θ), where ϕ(s, a) is a bit vector. ZDD cannot be combined until the exemplary embodiments focus on this specific case, that is, the specific action value function Q(s, a; w, θ)=w·ϕ(s, a)+f (s; θ). Moreover, it is noted that decision diagrams are often combined with planning, rather than reinforcement learning, where the environment is assumed to be known. Thus, the combination of Fitted Q-Iteration with the ZDD in offline RL focused on the specific action value function is unique.

In alternative embodiments, instead of multiple ZDDs, a single ZDDs can be employed.

For any set of bit vectors B, let ZDD(B) be the ZDD representing the set.

In the single ZDD embodiment, the exemplary embodiments construct ZDD for each s′ of (s, a, r, s′)∈ [symbol] , where:

ZDD({ϕ(s′, a′)|a′∈ [symbol] (s′)}), resulting in | [symbol] | ZDDs.

In another embodiment, the methods can construct a single ZDD for D as follows:

Let 1^{D} ┌{0,1}^{D} be the set of D-dimensional one-hot vectors, let η: [symbol] ′( [symbol] )→ [symbol] be a one-to-one mapping that assigns a one-hot vector to each state s∈ [symbol] ′( [symbol] ), where [symbol] ′( [symbol] ) denotes the set of resulting states in the data set [symbol] .

Then, the exemplary embodiments can construct:

ZDD({[ϕ(s′, a′)η(s′)]|a′∈ [symbol] (s′), s′∈ [symbol] ′( [symbol] )}), resulting in a single ZDD.

For any s′∈ [symbol] ′( [symbol] ), the exemplary embodiments can extract ZDD({ϕ(s′, a′)|a′∈ [symbol] (s′)}) from the single ZDD by a ZDD operation.

Therefore, the maximization step can be executed by first extracting the sub-ZDD.

In further alternative embodiments, binary decision diagrams (BDDs) can be substituted for ZDDs, because they also support the maximization operation.

In even further alternative embodiments, ZDD construction consumes much memory even if the resultant ZDD is small. Therefore, in one example, ZDD for a fixed data set D can be pre-computed in a computer with large main memory, and the resultant ZDD can be then transferred to a local computer with small memory for successive processes.

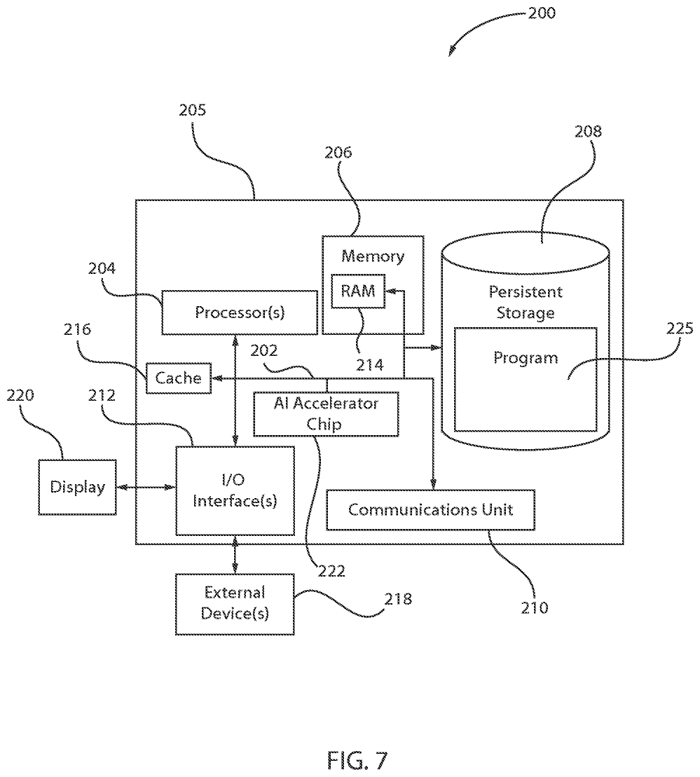

FIG. 7 is a block/flow diagram of an exemplary processing system for employing the Fitted Q-Iteration with the ZDD, in accordance with an embodiment of the present invention.

FIG. 7 depicts a block diagram of components of system 200, which includes computing device 205. It should be appreciated that FIG. 7 provides only an illustration of one implementation and does not imply any limitations with regard to the environments in which different embodiments can be implemented. Many modifications to the depicted environment can be made.

Computing device 205 includes communications fabric 202, which provides communications between computer processor(s) 204, memory 206, persistent storage 208, communications unit 210, and input/output (I/O) interface(s) 212. Communications fabric 202 can be implemented with any architecture designed for passing data and/or control information between processors (such as microprocessors, communications and network processors, etc.), system memory, peripheral devices, and any other hardware components within a system. For example, communications fabric 202 can be implemented with one or more buses.

Memory 206, cache memory 216, and persistent storage 208 are computer readable storage media. In this embodiment, memory 206 includes random access memory (RAM) 214. In another embodiment, the memory 206 can be flash memory. In general, memory 206 can include any suitable volatile or non-volatile computer readable storage media.

In some embodiments of the present invention, program 225 is included and operated by AI accelerator chip 222 as a component of computing device 205. In other embodiments, program 225 is stored in persistent storage 208 for execution by AI accelerator chip 222 (to implement Fitted Q-Iteration with the ZDD) in conjunction with one or more of the respective computer processors 204 via one or more memories of memory 206. In this embodiment, persistent storage 208 includes a magnetic hard disk drive. Alternatively, or in addition to a magnetic hard disk drive, persistent storage 208 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 208 can also be removable. For example, a removable hard drive can be used for persistent storage 208. Other examples include optical and magnetic disks, thumb drives, and smart cards that are inserted into a drive for transfer onto another computer readable storage medium that is also part of persistent storage 208.

Communications unit 210, in these examples, provides for communications with other data processing systems or devices, including resources of distributed data processing environment. In these examples, communications unit 210 includes one or more network interface cards. Communications unit 210 can provide communications through the use of either or both physical and wireless communications links. Deep learning program 225 can be downloaded to persistent storage 208 through communications unit 210.

I/O interface(s) 212 allows for input and output of data with other devices that can be connected to computing system 200. For example, I/O interface 212 can provide a connection to external devices 218 such as a keyboard, keypad, a touch screen, and/or some other suitable input device. External devices 218 can also include portable computer readable storage media such as, for example, thumb drives, portable optical or magnetic disks, and memory cards.

Display 220 provides a mechanism to display data to a user and can be, for example, a computer monitor.

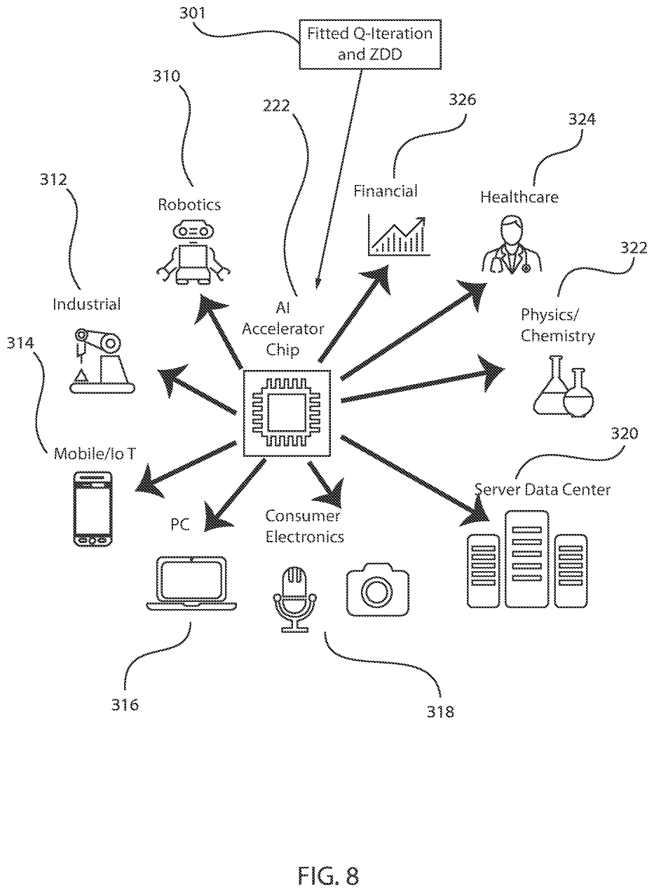

FIG. 8 illustrates practical applications for employing the Fitted Q-Iteration with the ZDD via an artificial intelligence (AI) accelerator chip, in accordance with an embodiment of the present invention.

The artificial intelligence (AI) accelerator chip 222 can implement the Fitted Q-Iteration with the ZDD 301, and can be used in a wide variety of practical applications, including, but not limited to, robotics 310, industrial applications 312, mobile or Internet-of-Things (IoT) 314, personal computing 316, consumer electronics 318, server data centers 320, physics and chemistry applications 322, healthcare applications 324, and financial applications 326.

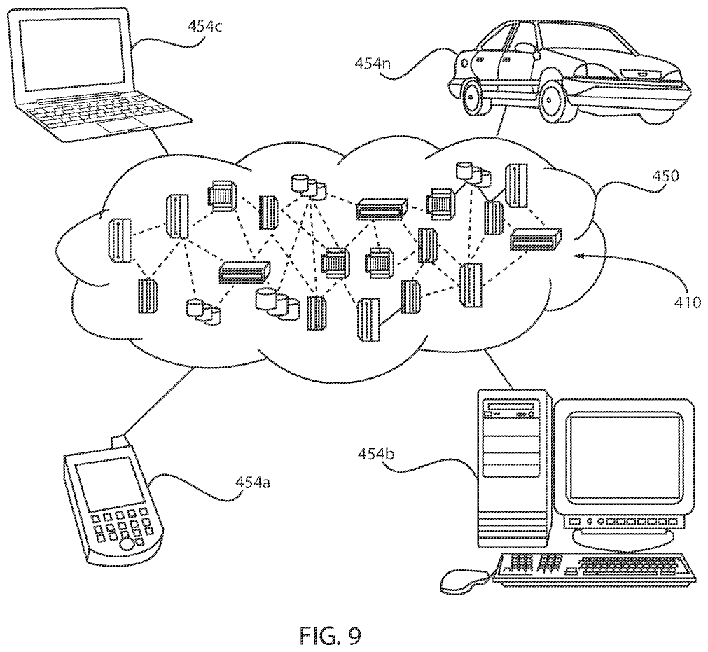

FIG. 9 is a block/flow diagram of an exemplary cloud computing environment, in accordance with an embodiment of the present invention.

It is to be understood that although this invention includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model can include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but can be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It can be managed by the organization or a third party and can exist on-premises or off-premises.

Community cloud: the cloud infrastructure is shared by several organizations and supports a specific community that has shared concerns (e.g., mission, security requirements, policy, and compliance considerations). It can be managed by the organizations or a third party and can exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 9, illustrative cloud computing environment 450 is depicted for enabling use cases of the present invention. As shown, cloud computing environment 450 includes one or more cloud computing nodes 410 with which local computing devices used by cloud consumers, such as, for example, personal digital assistant (PDA) or cellular telephone 454A, desktop computer 454B, laptop computer 454C, and/or automobile computer system 454N can communicate. Nodes 410 can communicate with one another. They can be grouped (not shown) physically or virtually, in one or more networks, such as Private, Community, Public, or Hybrid clouds as described hereinabove, or a combination thereof. This allows cloud computing environment 450 to offer infrastructure, platforms and/or software as services for which a cloud consumer does not need to maintain resources on a local computing device. It is understood that the types of computing devices 454A-N shown in FIG. 9 are intended to be illustrative only and that computing nodes 410 and cloud computing environment 450 can communicate with any type of computerized device over any type of network and/or network addressable connection (e.g., using a web browser).

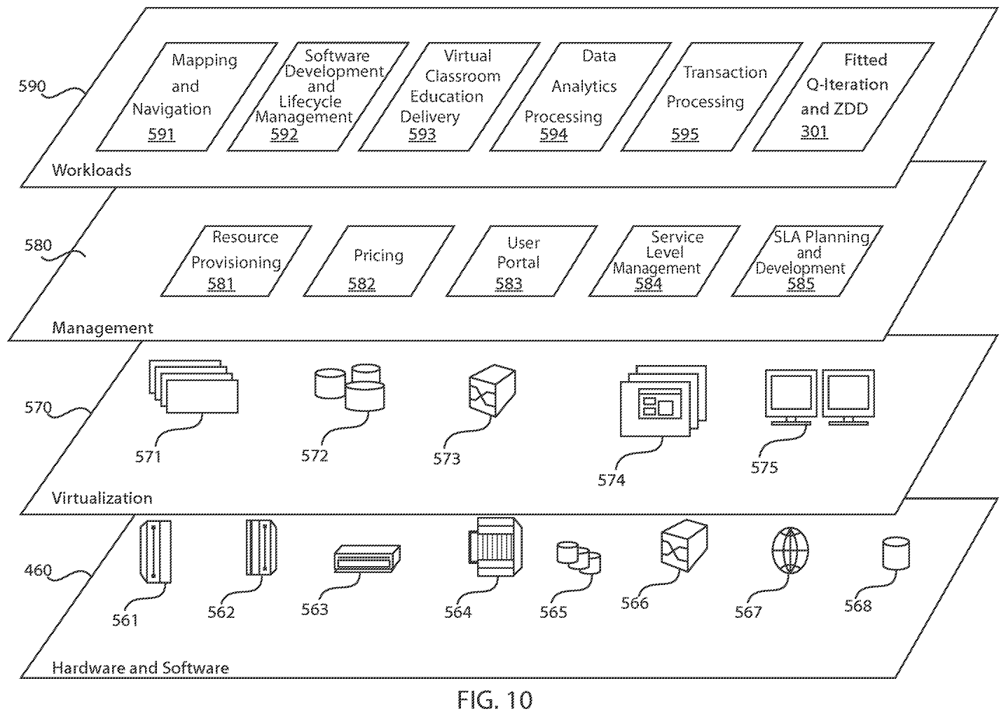

FIG. 10 is a schematic diagram of exemplary abstraction model layers, in accordance with an embodiment of the present invention. It should be understood in advance that the components, layers, and functions shown in FIG. 10 are intended to be illustrative only and embodiments of the invention are not limited thereto. As depicted, the following layers and corresponding functions are provided:

Hardware and software layer 560 includes hardware and software components. Examples of hardware components include: mainframes 561; RISC (Reduced Instruction Set Computer) architecture based servers 562; servers 563; blade servers 564; storage devices 565; and networks and networking components 566. In some embodiments, software components include network application server software 567 and database software 568.

Virtualization layer 570 provides an abstraction layer from which the following examples of virtual entities can be provided: virtual servers 571; virtual storage 572; virtual networks 573, including virtual private networks; virtual applications and operating systems 574; and virtual clients 575.

In one example, management layer 580 can provide the functions described below. Resource provisioning 581 provides dynamic procurement of computing resources and other resources that are utilized to perform tasks within the cloud computing environment. Metering and Pricing 582 provide cost tracking as resources are utilized within the cloud computing environment, and billing or invoicing for consumption of these resources. In one example, these resources can include application software licenses. Security provides identity verification for cloud consumers and tasks, as well as protection for data and other resources. User portal 583 provides access to the cloud computing environment for consumers and system administrators. Service level management 584 provides cloud computing resource allocation and management such that required service levels are met. Service Level Agreement (SLA) planning and fulfillment 585 provide pre-arrangement for, and procurement of, cloud computing resources for which a future requirement is anticipated in accordance with an SLA.

Workloads layer 590 provides examples of functionality for which the cloud computing environment can be utilized. Examples of workloads and functions which can be provided from this layer include: mapping and navigation 541; software development and lifecycle management 592; virtual classroom education delivery 593; data analytics processing 594; transaction processing 595; and Fitted Q-Iteration with the ZDD 301.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory, a read-only memory, an erasable programmable read-only memory (EPROM or Flash memory), a static random access memory, a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions can be provided to at least one processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks or modules. These computer readable program instructions can also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks or modules.

The computer readable program instructions can also be loaded onto a computer, other programmable data processing apparatus, or other device to cause a series of operational blocks/steps to be performed on the computer, other programmable apparatus or other device to produce a computer implemented process, such that the instructions which execute on the computer, other programmable apparatus, or other device implement the functions/acts specified in the flowchart and/or block diagram block or blocks or modules.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This can be extended, as readily apparent by one of ordinary skill in this and related arts, for as many items listed.

Having described preferred embodiments of a method for faster Fitted Q-iteration using a zero-suppressed decision diagram (which are intended to be illustrative and not limiting), it is noted that modifications and variations can be made by persons skilled in the art in light of the above teachings. It is therefore to be understood that changes may be made in the particular embodiments described which are within the scope of the invention as outlined by the appended claims. Having thus described aspects of the invention, with the details and particularity required by the patent laws, what is claimed and desired protected by Letters Patent is set forth in the appended claims.

1. A computer-implemented method for estimating a state-action value function for a Fitted Q-iteration, the method comprising:
obtaining a set of tuples D and a discount factor γ, each of the set of tuples including a state s, an action a, a reward r, and a resulting state s′;
constructing a zero-suppressed decision diagram (ZDD) of feature vectors {ϕ(s′, a ′)|a ′∈ [symbol] (s′)} for each of the resulting states s′ of the set of tuples, where the feature vector ϕ(s, a) is a sparse bit vector {0,1}^{D} and [symbol] (s′) is the set of actions applicable at state s′;
updating parameters w∈ [symbol] ^{D}, θ of a state-action value function Q(s, a; w, θ); and
repeating the updating step a predetermined times by incrementing t.

2. The computer-implemented method of claim 1, wherein the updating of the parameters is computed by:
$$( {w}_{t + 1} , {θ}_{t + 1} ) = {argmin}_{θ} {\sum }_{( s , a , {s}^{′} , r )} {[ Q ( s , a ; w , θ ) - ( r + γ \cdot {max}_{{a}^{′} ∈ A ( {s}^{′} )} Q ( {s}^{′} , {a}^{′} ; {w}_{t} , {θ}_{t} ) ) ]}^{2} , where Q ( s , a ; w , θ ) = w \cdot ϕ ( s , a ) + f ( s ; θ ) , \\ {max}_{{a}^{′} ∈ A ( {s}^{′} )} Q ( {s}^{′} , {a}^{′} ; {w}_{t} , {θ}_{t} ) = f ( {s}^{′} ; {θ}_{t} ) + {max}_{{a}^{′} ∈ A ( {s}^{′} )} {w}_{t} \cdot ϕ ( {s}^{′} , {a}^{′} ) \\ and {max}_{{a}^{′} ∈ A ( {s}^{′} )} {w}_{t} \cdot ϕ ( {s}^{′} , {a}^{′} )$$
is computed with the ZDD.

3. The computer-implemented method of claim 1, wherein the Fitted Q-iteration with ZDD is employed for computational material discovery for generating new molecular structures satisfying target property values.

4. The computer-implemented method of claim 1, wherein the state is a current molecule, the action is a chemical reaction, and the reward is a property to be maximized.

5. The computer-implemented method of claim 4, wherein the chemical reaction yields a plurality of candidates.

6. The computer-implemented method of claim 5, wherein a candidate of the plurality of candidates having a lowest synthetic accessibility score is selected as a product of the chemical reaction.

7. The computer-implemented method of claim 1, wherein the Fitted Q-iteration with ZDD is employed in offline reinforcement learning.

8. A computer program product for estimating a state-action value function for a Fitted Q-iteration, the computer program product comprising a computer readable storage medium having program instructions embodied therewith, the program instructions executable by a computer to cause the computer to:
obtain a set of tuples D and a discount factor γ, each of the set of tuples including a state s, an action a, a reward r, and a resulting state s′;
construct a zero-suppressed decision diagram (ZDD) of feature vectors {ϕ(s′, a ′)|a ′∈ [symbol] (s′)} for each of the resulting states s′ of the set of tuples, where the feature vector ϕ(s, a) is a sparse bit vector {0,1}^{D} and [symbol] (s′) is the set of actions applicable at state s′;
update parameters w∈ [symbol] ^{D}, θ of a state-action value function Q(s, a; w, θ); and
repeat the update step a predetermined times by incrementing t.

9. The computer program product of claim 8, wherein the update of the parameters is computed by:
$$( {w}_{t + 1} , {θ}_{t + 1} ) = {argmin}_{θ} {\sum }_{( s , a , {s}^{′} , r )} {[ Q ( s , a ; w , θ ) - ( r + γ \cdot {max}_{{a}^{′} ∈ A ( {s}^{′} )} Q ( {s}^{′} , {a}^{′} ; {w}_{t} , {θ}_{t} ) ) ]}^{2} , \\ where Q ( s , a ; w , θ ) = w \cdot ϕ ( s , a ) + f ( s ; θ ) , \\ {max}_{{a}^{′} ∈ A ( {s}^{′} )} Q ( {s}^{′} , {a}^{′} ; {w}_{t} , {θ}_{t} ) = f ( {s}^{′} ; {θ}_{t} ) + {max}_{{a}^{′} ∈ A ( {s}^{′} )} {w}_{t} \cdot ϕ ( {s}^{′} , {a}^{′} ) \\ and {max}_{{a}^{′} ∈ A ( {s}^{′} )} {w}_{t} \cdot ϕ ( {s}^{′} , {a}^{′} )$$
is computed with the ZDD.

10. The computer program product of claim 8, wherein the Fitted Q-iteration with ZDD is employed for computational material discovery for generating new molecular structures satisfying target property values.

11. The computer program product of claim 8, wherein the state is a current molecule, the action is a chemical reaction, and the reward is a property to be maximized.

12. The computer program product of claim 11, wherein the chemical reaction yields a plurality of candidates.

13. The computer program product of claim 12, wherein a candidate of the plurality of candidates having a lowest synthetic accessibility score is selected as a product of the chemical reaction.

14. The computer program product of claim 8, wherein the Fitted Q-iteration with ZDD is employed in offline reinforcement learning.

15. A system for estimating a state-action value function for a Fitted Q-iteration, the system comprising:
a memory; and
one or more processors in communication with the memory configured to:
obtain a set of tuples D and a discount factor γ, each of the set of tuples including a state s, an action a, a reward r, and a resulting state s′;
construct a zero-suppressed decision diagram (ZDD) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′;
update parameters w∈ [symbol] ^{D}, θ of a state-action value function Q(s, a; w, θ); and
repeat the update step a predetermined times by incrementing t.

16. The system of claim 15, wherein the update of the parameters is computed by:
$$( {w}_{t + 1} , {θ}_{t + 1} ) = {argmin}_{θ} {\sum }_{( s , a , {s}^{′} , r )} {[ Q ( s , a ; w , θ ) - ( r + γ \cdot {max}_{{a}^{′} ∈ A ( {s}^{′} )} Q ( {s}^{′} , {a}^{′} ; {w}_{t} , {θ}_{t} ) ) ]}^{2} , \\ where Q ( s , a ; w , θ ) = w \cdot ϕ ( s , a ) + f ( s ; θ ) , \\ {max}_{{a}^{′} ∈ A ( {s}^{′} )} Q ( {s}^{′} , {a}^{′} ; {w}_{t} , {θ}_{t} ) = f ( {s}^{′} ; {θ}_{t} ) + {max}_{{a}^{′} ∈ A ( {s}^{′} )} {w}_{t} \cdot ϕ ( {s}^{′} , {a}^{′} ) \\ and {max}_{{a}^{′} ∈ A ( {s}^{′} )} {w}_{t} \cdot ϕ ( {s}^{′} , {a}^{′} )$$
is computed with the ZDD.

17. The system of claim 15, wherein the Fitted Q-iteration with ZDD is employed for computational material discovery for generating new molecular structures satisfying target property values.

18. The system of claim 15, wherein the state is a current molecule, the action is a chemical reaction, and the reward is a property to be maximized.

19. The system of claim 18, wherein the chemical reaction yields a plurality of candidates.

20. The system of claim 19, wherein a candidate of the plurality of candidates having a lowest synthetic accessibility score is selected as a product of the chemical reaction.

21. The system of claim 15, wherein the Fitted Q-iteration with ZDD is employed in offline reinforcement learning.

22. A computer-implemented method for estimating a state-action value function for a Fitted Q-iteration, the method comprising:
obtaining a set of tuples D and a discount factor γ, each of the set of tuples including a state s, an action a, a reward r, and a resulting state s′;
constructing a binary decision diagram (BDD) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′;
updating parameters w∈ [symbol] ^{D}, θ of a state-action value function Q(s, a; w, θ); and
repeating the updating step a predetermined times by incrementing t.

23. The computer-implemented method of claim 22, wherein the updating of the parameters is computed by:
$$( {w}_{t + 1} , {θ}_{t + 1} ) = {argmin}_{θ} {\sum }_{( s , a , {s}^{′} , r )} {[ Q ( s , a ; w , θ ) - ( r + γ \cdot {max}_{{a}^{′} ∈ A ( {s}^{′} )} Q ( {s}^{′} , {a}^{′} ; {w}_{t} , {θ}_{t} ) ) ]}^{2} , \\ where Q ( s , a ; w , θ ) = w \cdot ϕ ( s , a ) + f ( s ; θ ) , \\ {max}_{{a}^{′} ∈ A ( {s}^{′} )} Q ( {s}^{′} , {a}^{′} ; {w}_{t} , {θ}_{t} ) = f ( {s}^{′} ; {θ}_{t} ) + {max}_{{a}^{′} ∈ A ( {s}^{′} )} {w}_{t} \cdot ϕ ( {s}^{′} , {a}^{′} ) \\ and {max}_{{a}^{′} ∈ A ( {s}^{′} )} {w}_{t} \cdot ϕ ( {s}^{′} , {a}^{′} )$$
is computed with the BDD.

24. A computer-implemented method for estimating a state-action value function for a Fitted Q-iteration, the method comprising:
obtaining a set of tuples D and a discount factor γ, each of the set of tuples including a state s, an action a, a reward r, and a resulting state s′;
constructing a plurality of zero-suppressed decision diagrams (ZDDs) of feature vectors {ϕ(s′, a′)|a′∈ [symbol] (s′)} for each of the resulting states s′ of the set of tuples, where the feature vector ϕ(s, a) is a sparse bit vector {0,1}^{D} and [symbol] (s′) is the set of actions applicable at state s′;
updating parameters w∈ [symbol] ^{D}, θ of a state-action value function Q(s, a; w, θ); and
repeating the updating step a predetermined times by incrementing t.

25. The computer-implemented method of claim 24, wherein the updating of the parameters is computed by:
$$( {w}_{t + 1} , {θ}_{t + 1} ) = {argmin}_{θ} {\sum }_{( s , a , {s}^{′} , r )} {[ Q ( s , a ; w , θ ) - ( r + γ \cdot {max}_{{a}^{′} ∈ A ( {s}^{′} )} Q ( {s}^{′} , {a}^{′} ; {w}_{t} , {θ}_{t} ) ) ]}^{2} , \\ where Q ( s , a ; w , θ ) = w \cdot ϕ ( s , a ) + f ( s ; θ ) , \\ {max}_{{a}^{′} ∈ A ( {s}^{′} )} Q ( {s}^{′} , {a}^{′} ; {w}_{t} , {θ}_{t} ) = f ( {s}^{′} ; {θ}_{t} ) + {max}_{{a}^{′} ∈ A ( {s}^{′} )} {w}_{t} \cdot ϕ ( {s}^{′} , {a}^{′} ) \\ and {max}_{{a}^{′} ∈ A ( {s}^{′} )} {w}_{t} \cdot ϕ ( {s}^{′} , {a}^{′} )$$
is computed with the plurality of ZDDs.
